(19) United States
(12) Patent Application Publication
(10) Pub. No.: US 2023/0049827 A1
(43) Pub. Date: Feb. 16, 2023

(54) PHYSIOLOGICAL SIGNAL MONITORING APPARATUS

(71) Applicant: Chih-Liang WU, Taipei (TW)

(72) Inventor: Chih-Liang WU, Taipei (TW)

(21) Appl. No.: 17/785,645

(22) PCT Filed: Dec. 29, 2020
(86) PCT No.: PCT/CN2020/140723

§ 371 Date: Jun. 15, 2022

(30) Foreign Application Priority Data
Dec. 31, 2019 (CN) 201911409123.X

(51) Int. Cl. A61B 5/259; A61B 5/332; A61B 5/282

(52) U.S. Cl. CPC A61B 5/259; A61B 5/332; A61B 5/282; A61B 2562/18

(57) ABSTRACT

Provided is a physiological signal monitoring apparatus, including: a waterproof housing, a base, an electrocardiogram signal processing unit, an upper patch layer, a lower patch layer, and at least two electrodes, wherein the electrocardiogram signal processing unit is accommodated between the waterproof housing and the base; and the electrodes are located between the upper patch layer and the lower patch layer, and are electrically coupled to the electrocardiogram signal processing unit, so as to sense electrocardiogram signals for the electrocardiogram signal processing unit to process. Due to the special design of the upper and lower patch layers, external water vapor or moisture can be effectively prevented from directly entering an area which is in contact with skin, such that the physiological signal monitoring apparatus can be firmly attached to the skin, thereby prolonging the usage time thereof, reducing the replacement frequency of same, and specifically achieving the aim of long-term monitoring and sensing of electrocardiogram signals. The monitoring apparatus is particularly convenient and helpful for patients suffering from cardiovascular diseases and requiring long-term recording of electrocardiogram signals.

### CROSS-REFERENCE TO RELATED APPLICATION

This application claims the priority of Chinese patent application No. 201911409123.X, filed on Dec. 31, 2019, which is incorporated herewith by reference.

### BACKGROUND OF THE INVENTION

### 1. Field of the Invention

The present invention relates to a physiological signal monitoring apparatus, and more particularly, to a physiological signal monitoring apparatus in which an electrocardiogram signal processing unit is accommodated between a waterproof housing and a base; and electrodes are located between an upper patch layer and a lower patch layer, and are electrically coupled to the electrocardiogram signal processing unit, so as to sense electrocardiogram signals for the electrocardiogram signal processing unit to process. Due to the special design of the upper and lower patch layers, external water vapor or moisture can be effectively prevented from directly entering an area which is in contact with skin, such that the physiological signal monitoring apparatus can be firmly attached to the skin, thereby prolonging the usage time thereof, reducing the replacement frequency of same, and specifically achieving the aim of long-term monitoring and sensing of electrocardiogram signals. The monitoring apparatus is particularly convenient and helpful for patients suffering from cardiovascular diseases and requiring long-term recording of electrocardiogram signals.

### 2. The Prior Arts

As well known in the art, specific physiological electrical signals on the surface of the body will be generated as the physiological activities of the body occur. With the continuous progress of medical technology, the relationship between many diseases and physiological signals has been studied in detail.

At present, measuring patches can be used to be attached on specific positions on the surface of the body, such as head, chest, abdomen, back, and limbs, and various physiological electrical signals, such as electroencephalogram signal (EEG), electromyogram signal (EMG), electroneurogram signal (ENG), electroretinogram signal (ERG), electrogastrogram signal (EGG), electroneuromyogram signal (ENMG), electrocorticogram signal (ECoG), electrooculogram signal (EOG), and electronystagmogram (ENG), etc., can be measured in a noninvasive manner. In particular, abnormal blood pressure and heartbeat often relate to cardiovascular disease. Therefore, it is quite common to use a physiological signal measuring apparatus to measure heart rate-related information, which can be used as an index reference data for health status.

In the related arts, general electrical signal measuring patches are manufactured by disposing a conductive metal sheet and a label on an attachment layer of foam adhesive, attaching an electrode sheet to the bottom of the foam adhesive, and then attaching a conductive adhesive under the electrode sheet. In addition, the conductive metal sheet is electrically coupled to the electrode sheet. During use, it is necessary that the conductive adhesive is in direct contact with the surface of the human body, and then the conductive metal sheet is connected to an external device through a signal line. The electrical signals on the surface of the human body can be measured by the electrode sheet via the conductive adhesive, and then transmitted to the external device by the conductive metal sheet, so as to conduct specific analysis and display relevant physiological signal waveforms, data and status.

The relevant manufacturers have tried their best to produce the measuring patches as a thin sheet, which can be directly attached to the human body and can be easily peeled off, and thus is very convenient to use. However, the related arts are disadvantageous in that each patch can only measure the electrical signals at a single position. In practical application, it is often required to conduct measurements at multiple positions, so that patches and connecting lines are distributed all over the body, thereby the patches and connecting lines are prone to be pulled apart, which causes the medical staff or patients trouble especially in moving.

Therefore, some manufacturers have developed portable measuring patches, which can be directly attached to the body for measurement without any connecting line, thereby significantly improving the convenience of operation. However, such measuring patches are not waterproof, so that they are prone to fall off after the user sweats, such as during exercise; especially, they are not suitable for use during a bath. Therefore, the user must take off the measuring patch before exercising or taking a bath, which causes inconvenience of use. In addition, such measuring patches are only suitable for use in a motionless state and cannot be used for a long time. Therefore, not only the usage environment is limited, but the usage time is not long enough.

Furthermore, the monitoring patches of the related arts have poor air permeability and moisture permeability, so that sweat accumulation is prone to occur, which causes sultry and damp feelings, and even allergy, inflammation, and itch on the surface of the body, thereby resulting discomfort.

Therefore, there is a great need for an innovative physiological signal monitoring apparatus in which an electrocardiogram signal processing unit is accommodated between a waterproof housing and a base; and electrodes are located between an upper patch layer and a lower patch layer, and are electrically coupled to the electrocardiogram signal processing unit, so as to sense electrocardiogram signals for the electrocardiogram signal processing unit to process. Due to the special design of the upper and lower patch layers, external water vapor or moisture can be effectively prevented from directly entering an area which is in contact with skin, such that the physiological signal monitoring apparatus can be firmly attached to the skin, thereby prolonging the usage time thereof, reducing the replacement frequency of same, and specifically achieving the aim of long-term monitoring and sensing of electrocardiogram signals. The monitoring apparatus is particularly convenient and helpful for patients suffering from cardiovascular diseases and requiring long-term recording of electrocardiogram signals. According to this, all the problems of the above-mentioned related arts are solved.

### SUMMARY OF THE INVENTION

A primary objective of the present invention is to provide a physiological signal monitoring apparatus, which comprises a waterproof housing, a base, an electrocardiogram signal processing unit, an upper patch layer, a lower patch layer, and at least two electrodes, and is used for long-term monitoring of physiological information such as electrocardiogram signals.

Specifically, the waterproof housing is provided with a bottom opening, the base is located at a bottom of the waterproof housing, and the base and the bottom opening jointly define a closed waterproof accommodation space for accommodating the electrocardiogram signal processing unit.

The upper patch layer has an upper surface and a lower surface, wherein the lower surface of the upper patch layer is sticky, the waterproof housing is located at the upper surface of the upper patch layer, and the base is located at the lower surface of the upper patch layer; particularly, a middle area of the upper patch layer is sandwiched by the waterproof housing and the base.

The lower patch layer has an upper surface and a lower surface, wherein the lower surface of the lower patch layer is sticky for being attached to a skin, and the lower patch layer is located under the upper patch layer and the base, and wherein the horizontal dimension of the lower patch layer is smaller than that of the upper patch layer.

Said at least two electrodes are located between the upper patch layer and the lower patch layer without contacting with each other. Each of the electrodes is electrically coupled to the electrocardiogram signal processing unit, and penetrates through a joint between the waterproof housing and the base and extends outward in an extension direction, so as to sense an electrocardiogram signal generated by activity of a heart, and transmit the electrocardiogram signal to the electrocardiogram signal processing unit.

In addition, all the joint between the waterproof housing and the base overlap the upper patch layer in a vertical direction, and a plurality of parts of the joint also overlap the at least two electrodes and the lower patch layer in the vertical direction, so as to strengthen the structure of the overall apparatus, thereby resisting the user's pulling at the apparatus during actions.

As a whole, the above-mentioned physiological signal monitoring apparatus can be attached to a skin of the human body to sense electrocardiogram signals. Due to the special design of the upper and lower patch layers, external water vapor or moisture can be effectively prevented from directly entering an area which is in contact with skin, such that the physiological signal monitoring apparatus of the present invention can be firmly attached to the skin, thereby prolonging the usage time thereof, reducing the replacement frequency of same, and specifically achieving the aim of long-term monitoring and sensing of electrocardiogram signals. The monitoring apparatus is particularly convenient and helpful for patients suffering from cardiovascular diseases and requiring long-term recording of electrocardiogram signals.

Furthermore, a further objective of the present invention is to provide a physiological signal monitoring apparatus, which comprises a waterproof housing, an electrocardiogram signal processing unit, an upper patch layer, a lower patch layer, at least two electrodes, and a base, and is used to be attached to the skin for long-term monitoring of physiological signals.

The waterproof housing is a closed waterproof housing itself, and the electrocardiogram signal processing unit is disposed in the waterproof housing. The upper patch layer has an upper surface and a lower surface, wherein the upper surface of the upper patch layer is provided with a plurality of macroscopic perforation structures providing the air permeability and the moisture permeability, and the lower surface of the upper patch layer is sticky for being attached to the skin. The lower patch layer has an upper surface and a lower surface, wherein the lower surface of the lower patch layer is sticky for being attached to the skin.

The base has an upper surface and a lower surface. The base is disposed between the upper patch layer and the lower patch layer, or on the upper patch layer, or under the lower patch layer, so as to fix and carry the waterproof housing. The at least two electrodes are located between the upper patch layer and the lower patch layer without contacting with each other, wherein each of the electrodes is electrically coupled to the electrocardiogram signal processing unit. In addition, at least one gap is present between the bottom of the waterproof housing and the upper surface of the upper patch layer. Therefore, the bottom of the waterproof housing and the upper patch layer are not completely in contact with each other in fact, but in contact with each other with at least one gap therebetween or when being separated by a specific distance, so as to facilitate drainage of moisture.

In the above-mentioned physiological signal monitoring apparatus, the waterproof housing being a closed structure itself can be combined with the base and then fixed. In particular, the bottom of the waterproof housing is not completely in contact with the upper patch layer, while the base and the waterproof housing can be combined with each other through hooks and protruding structures of the base. In particular, the hooks are perpendicular to the upper patch layer, such that a gap is formed between the surface of the upper lower patch layer and the bottom of the waterproof housing, which facilitates air permeation and drainage of moisture.

### BRIEF DESCRIPTION OF THE DRAWINGS

FIG. 1 is a schematic perspective view of a physiological signal monitoring apparatus according to a first embodiment of the present invention.

FIG. 2 is a side view of the physiological signal monitoring apparatus according to the first embodiment of the present invention.

FIG. 3 is a side view of the physiological signal monitoring apparatus according to the first embodiment of the present invention observed from another angle of view.

FIG. 4 is a schematic perspective view of a physiological signal monitoring apparatus according to a second embodiment of the present invention.

FIG. 5 is a part sectioned view of the physiological signal monitoring apparatus according to the second embodiment of the present invention.

FIG. 6 is a partial schematic perspective view of the physiological signal monitoring apparatus according to the second embodiment of the present invention.

FIG. 7 is another schematic diagram illustrating the physiological signal monitoring apparatus according to the second embodiment of the present invention.

### DETAILED DESCRIPTION OF THE PREFERRED EMBODIMENT

The following description provides a further understanding of the embodiments of the present invention in conjunction with the drawings and the reference numerals, in order that those skilled in the art can implement them after studying the present specification.

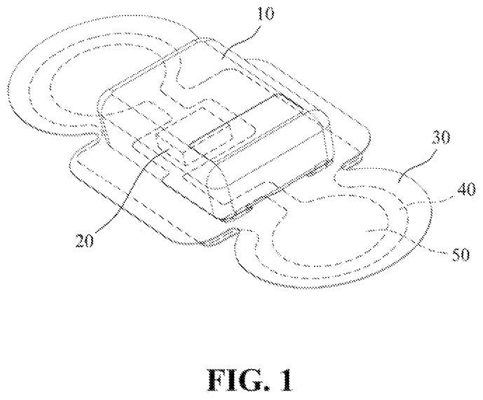

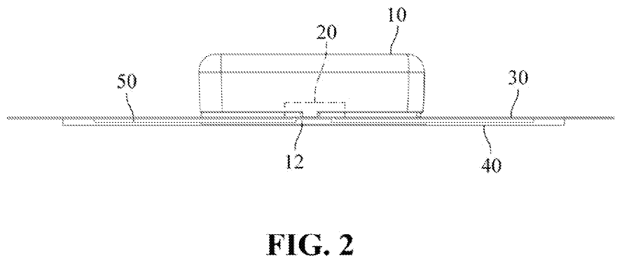

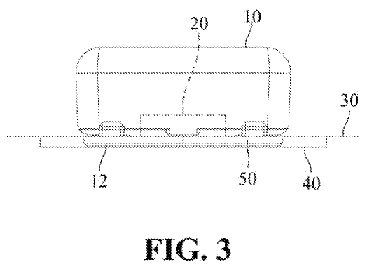

Referring to FIGS. 1, 2 and 3 simultaneously, which are a schematic perspective view, a side view, and a side view observed from another angle of view of a physiological signal monitoring apparatus according to a first embodiment of the present invention, respectively, wherein the angle of views of FIGS. 2 and 3 are perpendicular to each other. For example, FIG. 2 is a front view while FIG. 3 is a right side view. As shown in FIGS. 1, 2 and 3, the physiological signal monitoring apparatus according to the first embodiment of the present invention comprises a waterproof housing 10, a base 12, an electrocardiogram signal processing unit 20, an upper patch layer 30, a lower patch layer 40, and at least two electrodes 50, and is used for long-term monitoring of physiological information, especially electrocardiogram signals, but the present invention is not limited thereto.

Specifically, the waterproof housing 10 is provided with a bottom opening, the base 12 is located at a bottom of the waterproof housing 10, and the base 12 and the bottom opening jointly define a closed waterproof accommodation space in which the electrocardiogram signal processing unit 20 is disposed. In addition, the upper patch layer 30 has an upper surface and a lower surface, wherein the lower surface of the upper patch layer 30 is sticky, the waterproof housing 10 is located at the upper surface of the upper patch layer 30, and the base 12 is located at the lower surface of the upper patch layer 30; particularly, a middle area of the upper patch layer 30 is sandwiched by the waterproof housing 10 and the base 12.

In addition, the lower patch layer 40 has an upper surface and a lower surface, wherein the lower surface of the lower patch layer 40 is sticky for being attached to a skin, and the lower patch layer 40 is located under the upper patch layer 30 and the base 12. In particular, the horizontal dimension of the lower patch layer 40 is smaller than that of the upper patch layer 30, that is, the lower patch layer is completely covered and protected by the upper patch layer, so as to avoid the external water vapor from penetrating therein.

Further, the at least two electrodes 50 are located between the upper patch layer 30 and the lower patch layer 40 without contacting with each other, wherein each of the electrodes 50 is electrically coupled to the electrocardiogram signal processing unit 20, and penetrates through a joint between the waterproof housing 10 and the base 12 and extends outward in an extension direction. Therefore, each of the electrodes 50 can be used to sense the electrocardiogram signal generated by activity of a heart, and transmit the electrocardiogram signal to the electrocardiogram signal processing unit 20.

In particular, at least a part of the joint between the waterproof housing 10 and the base 12 overlap the upper patch layer 30 and the lower patch layer 40 in a vertical direction, while the remaining parts of the joint overlap only the upper patch layer 30 in the vertical direction.

More specifically, the lower patch layer 40 is disposed at a bottom of the base 12 and a bottom of the upper patch layer 30, so as to be attached and fixed to the skin; wherein a portion of the lower patch layer 40 disposed at the bottom of the base 12 and the remaining portion of the lower patch layer 40 disposed at the bottom of the upper patch layer 30 are partially located at different levels, or the portion of the lower patch layer 40 disposed at the bottom of the base 12 and the remaining portion of the lower patch layer 40 disposed at the bottom of the upper patch layer 30 are not integrally formed.

In another aspect, a strengthening layer (not shown in the drawings) is provided between the upper patch layer 30 and the lower patch layer 40, wherein the area of the strengthening layer is smaller than that of the upper patch layer 30, the at least two electrodes 50 are located between the upper patch layer 30 and the strengthening layer, and the strengthening layer is attached under the at least two electrodes 50, so as to enhance the tensile strength of the at least two electrodes.

The above-mentioned electrocardiogram signal processing unit 20 comprises a power storage unit and a power supply unit for storing power and supplying power for operation.

Furthermore, the bottom of the upper patch layer 30 and the bottoms of the lower patch layer 40 are completely covered with adhesive, wherein the lower patch layer 40 can be formed by laminating upper and lower portions respectively containing two materials with different water absorbency. Particularly, the water absorption rate of the upper portion is higher than that of the lower portion, and the material of the lower portion is in direct contact with the skin. In another aspect, the thickness of the lower patch layer 40 is greater than that of the upper patch layer 30. The upper patch layer 30 is waterproof, and a distance of the upper patch layer 30 extending horizontally outward from the waterproof housing 10 is greater than a distance of the lower patch layer 40 extending horizontally outward, such that the lower patch layer 40 is completely covered by the upper patch layer 30, so as to avoid the external water vapor from penetrating therein. Particularly, a contact area between the upper patch layer 30 and the skin when the upper patch layer 30 is attached to the skin is greater than an area where the at least two electrodes 50 are distributed.

Each of said electrodes 50 has an end portion extending outward, and the lower patch layer 40 is provided with at least two holes respectively corresponding to the end portions, wherein the end portions of the electrodes 50 are in contact with the skin through the holes, so as to sense the electrocardiogram signals. The holes of the lower patch layer 40 are closely engaged with corresponding end portions respectively to provide waterproof effect, wherein the holes and the corresponding end portions are closely engaged with each other respectively through a double-sided adhesive layer (not shown in the drawings) provided therebetween. In addition, a distance of the upper patch layer 30 extending horizontally outward from the waterproof housing 10 is greater than a distance of the at least two electrodes 50 extending horizontally outward, such that the at least two electrodes 50 are completely covered by the upper patch layer 30.

Each of the end portions of the at least two electrodes 50 may be an integrally formed conductive electrode, for example, may be made of copper, so as to be attached to the skin for sensing electrocardiogram signals; or each of the end portions of the at least two electrodes 50 may consist of the combination of a conductive electrode and a viscous conductive adhesive, wherein the conductive adhesive is used to be attached to the skin as a medium for transmitting electrical signals between the skin and the at least two electrodes 50. In addition, inside the waterproof housing 10, the at least two electrodes 50 are completely covered and sandwiched by the upper patch layer 30 and the lower patch layer 40; while the upper patch layer 30 is only provide with an opening at the end portion of each of the at least two electrodes 50 for electrically coupling to the electrocardiogram signal processing unit 20.

Further, the upper patch layer 30 is waterproof, and a portion of an outer edge of the upper patch layer 30 extending outward beyond the lower patch layer 40 forms a continuous and uninterrupted area, so as to be completely attached to the skin, thereby forming an area closed with respect to exterior to protect the bottom of the waterproof housing 10 and the lower patch layer 40. In addition, one of the at least two electrodes 50 only extends outward from an interior of the waterproof housing 10 to an area where the upper patch layer 30 and the lower patch layer 40 overlap with each other, but does not extend to an area where only the upper patch layer 30 is distributed.

As a whole, the physiological signal monitoring apparatus according to the first embodiment of the present invention can be attached to the skin of the human body to sense electrocardiogram signals. Due to the special design of the upper and lower patch layers, external water vapor or moisture can be effectively prevented from directly entering an area which is in contact with skin, such that the physiological signal monitoring apparatus of the present invention can be firmly attached to the skin, thereby prolonging the usage time thereof, reducing the replacement frequency of same, and specifically achieving the aim of long-term monitoring and sensing of electrocardiogram signals. The monitoring apparatus is particularly convenient and helpful for patients suffering from cardiovascular diseases and requiring long-term recording of electrocardiogram signals.

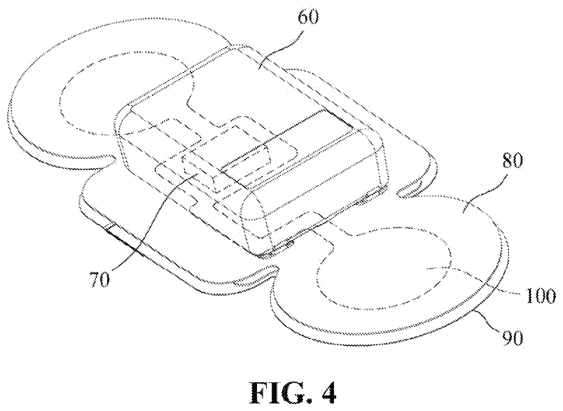

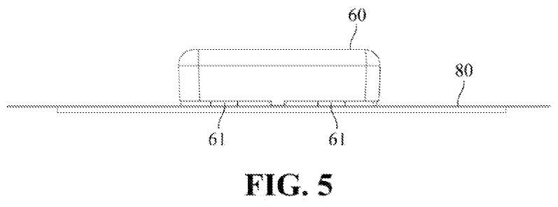

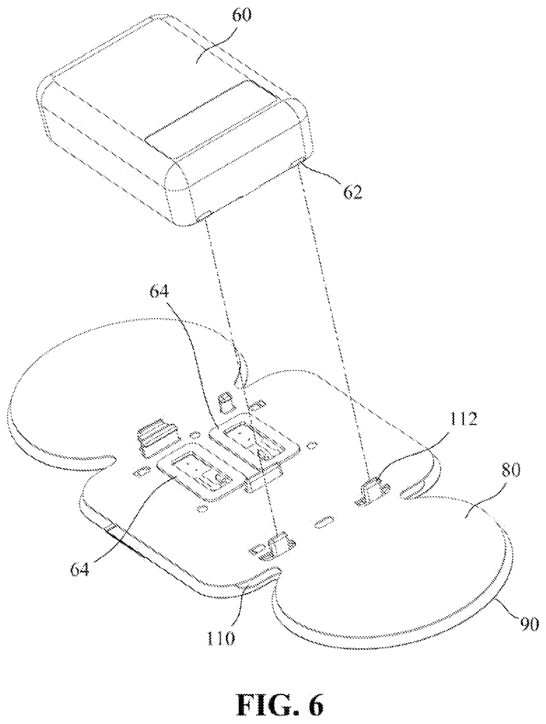

Hereinafter, referring to FIGS. 4, 5 and 6 simultaneously, wherein FIG. 4 is a schematic perspective view of a physiological signal monitoring apparatus according to a second embodiment of the present invention, FIG. 5 is a part sectioned view of the physiological signal monitoring apparatus according to the second embodiment of the present invention, and FIG. 6 is a partial schematic perspective view of the physiological signal monitoring apparatus according to the second embodiment of the present invention. As shown in FIGS. 4, 5 and 6, the physiological signal monitoring apparatus according to the second embodiment of the present invention comprises a waterproof housing 60, an electrocardiogram signal processing unit 70, an upper patch layer 80, a lower patch layer 90, at least two electrodes 100, and a base 110, and is used to be attached to the skin for long-term monitoring of physiological signals. However, it should be noted that FIG. 5 only shows the waterproof housing 60 and the upper patch layer 80 of the physiological signal monitoring apparatus according to the second embodiment, for illustrating the connection features therebetween, while FIG. 6 only shows the waterproof housing 60, the upper patch layer 80, the lower patch layer 90 and the base 110, wherein the electrocardiogram signal processing unit 70 and the electrodes 100 are not shown.

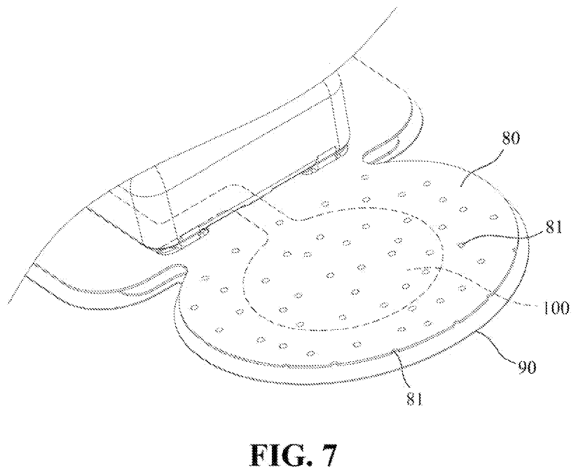

Specifically, the waterproof housing 60 is a closed waterproof housing; the electrocardiogram signal processing unit 70 is disposed in the waterproof housing 60 and has the functions of wirelessly transmitting signals and calculating the heart rate; and the upper patch layer 80 has an upper surface and a lower surface. As further shown in FIG. 7, a plurality of macroscopic perforation structures or perforations 81 are randomly distributed over the entire upper patch layer 80. Particularly, the perforations 81 distributed at the outer edge of the upper patch layer 80 can be notched perforations, while the perforations 81 distributed at the other portion on the upper patch layer 80 are intact perforations. The upper patch layer 80 can be, for example, made of non-woven fabric, woven fabric, or cotton fabric material, and these perforation structures or perforations 81 provide the air permeability and the moisture permeability, which can be used to increase the comfort of attachment. Particularly, the lower surface of the upper patch layer 80 is sticky for being attached and fixed to a skin, while the plurality of macroscopic perforation structures or perforations 81 may be distributed on the upper surface or the lower surface of the upper patch layer 80. In addition, the lower patch layer 90 has an upper surface and a lower surface, wherein the lower surface of the lower patch layer 90 is sticky for being attached to a skin and then fixed.

Furthermore, the base 110 has an upper surface and a lower surface. The base 110 is disposed between the upper patch layer 80 and the lower patch layer 90, or on the upper patch layer 80, or under the lower patch layer 90, and thus can be used for fixing and carrying the waterproof housing 60. The at least two electrodes 100 are located between the upper patch layer 80 and the lower patch layer 90 without contacting with each other, wherein each of the electrodes 100 is electrically coupled to the electrocardiogram signal processing unit 70.

In particular, a gap is present between the bottom of the waterproof housing 60 and the upper surface of the upper patch layer 80, wherein the gap can be not less than 0.1 mm. More specifically, the bottom of the waterproof housing 60 and the upper surface of the upper patch layer 80 or the bottom of the waterproof housing 60 and the base 110 are combined in a partially contacting manner; therefore, at least one gap 61 is present therebetween, as shown in FIG. 5, so as to facilitate evaporation of water vapor. The gap 60 can be a penetration gap, wherein the height of the gap 61 can also be not less than 0.1 mm. For example, the bottom of the waterproof housing 60 is provided with at least one protruding structure combined with the upper surface of the upper patch layer 80, such that the remaining part of the bottom of the waterproof housing 60 is suspended without contact with the upper surface of the upper patch layer 80.

Further, the upper patch layer 80 and the lower patch layer 90 are located in the same side areas as the at least two electrodes 100 and extend horizontally outward to form electrode side outer edges of the upper patch layer and electrode side outer edges of the lower patch layer, respectively, wherein the electrode side outer edges of the upper patch layer and the electrode side outer edges of the lower patch layer are aligned with each other, respectively.

Furthermore, the upper patch layer 80 extends horizontally beyond the lower patch layer 90 to form an outer edge of the upper patch layer, which can be attached to the skin, wherein the air permeability and the moisture permeability of the outer edge of the upper patch layer are greater than those of a portion where the upper patch layer 80 and the lower patch layer 90 are combined with each other; the thickness of the outer edge of the upper patch layer is less than that of the portion where the upper patch layer 80 and the lower patch layer 90 are combined with each other; and the color of the outer edge of the upper patch layer is different from, such as lighter than, that of the portion where the upper patch layer 80 and the lower patch layer 90 are combined with each other, which helps the user to clearly identify the deterioration state of the patch during use, especially when the outer edge of the patch layers may lift first during use. In addition, the transmittance of the outer edge of the upper patch layer is different from that of the portion where the upper patch layer 80 and the lower patch layer 90 are combined with each other, therefore, it is more possible to see through the background thereof such as the color of the skin, which helps the user to clearly identify the deterioration state of the patch during use, especially when the outer edge of the patch layers may lift first during use.

Furthermore, the lower patch layer 90 is provided with at least two holes corresponding to the end portions of the electrodes 100 extending outward, respectively, wherein the end portions are in contact with the skin through the holes for sensing the electrocardiogram signals. Furthermore, the end portions of the electrodes 100 can completely cover and be closely engaged with the corresponding holes of the lower patch layer 90 to generate waterproof effect. Therefore, the transmission of electrocardiogram signals will not be disturbed by sweat and shower; alternatively, the end portions of the electrodes 100 and the corresponding holes are closely engaged with each other through a double-sided adhesive; alternatively, the end portions of the electrodes 100 and the corresponding holes are closely engaged with each other by means of physical welding.

The lower patch layer 90 is provided with a plurality of perforation structures (not shown in the drawings) or perforations distributed over the entire lower patch layer 90, wherein the perforation structures are macroscopic and provide air permeability and moisture permeability. For example, the lower patch layer 90 can be made of non-woven fabrics. The perforations of the upper patch layer 80 and the perforations of the lower patch layer 90 may be not aligned with each other, such that the air permeability and moisture permeability of an area where the upper patch layer 80 and the lower patch layer 90 overlap with each other are lower than those of the non-overlapping area, which provides water blocking function, thereby can prevent the external moisture or water vapor from directly flushing the adhesive at the bottom of the upper patch layer 80 and the lower patch layer 90 and affecting the overall attachment performance. Particularly, the perforation structures of the upper patch layer 80 or the perforation structures of the lower patch layer 90 may be fine pores formed by interweaving in which each adjacent pores is arranged orderly. For example, the upper patch layer 80 or the lower patch layer 90 may be made of woven fabric or cotton fabric material.

The lower patch layer 90 can be a double-sided adhesive which is sticky on both sides, wherein the double-sided adhesive can also be a double-sided adhesive layer without substrate. The upper patch layer 80 and the lower patch layer 90 may also be made of soft materials, wherein the soft materials may include non-woven fabric materials, woven fabric materials, and fiber materials, etc. The upper patch layer 80 and the lower patch layer 90 may also be closely combined with each other by means of physical welding.

Further, the hardness of the base 110 is greater than that of the upper patch layer 80 and that of the lower patch layer 90, and the ductility of the base 110 is specially designed to be lower than that of the upper patch layer 80 and that of the lower patch layer 90. The base 110 may be further provided with a plurality of perforation structures, which can increase the air permeability and the moisture permeability during long-term attachment.

In addition, the area where the lower patch layer 90 is attached to the skin is greater than the area where the upper patch layer 80 is attached to the skin. The distances of the upper patch layer 80 and the lower patch layer 90 extending horizontally outward from the waterproof housing 60 are greater than a distance of the at least two electrodes 100 extending horizontally outward, respectively, such that the at least two electrodes 100 are completely covered by the upper patch layer 80 and the lower patch layer 90, which can prevent the signal transmission of the electrodes 100 from being disturbed by the external environment.

Each of the electrodes 100 has an upper surface and a lower surface, wherein the lower surfaces and the upper surfaces of the electrodes 100 have vertical conductive functions, and are used to contact an external conductive element (not shown in the drawings) provided extendedly from the waterproof housing 60 for electrically coupling to the electrocardiogram signal processing unit 70. Therefore, if it is required to use the physiological signal monitoring apparatus of the present invention repeatedly, the electrodes 100 or even the upper patch layer 80 and the lower patch layer 90 can be easily detached and replaced, so that it is very convenient to use.

The above-mentioned external conductive element can be embedded into the waterproof housing 60, wherein a part of the external conductive element can be disposed inside the waterproof housing 60 and electrically coupled to the electrocardiogram signal processing unit 70, while the other part of the external conductive element extends outward from the waterproof housing 60. Particularly, the height of the part extending outward is higher than that of the embedded part.

Further, the external conductive element may be a conductive connector with a spring structure, a conductive pillar, a conductive fabric, conductive pins, a conductive foam, or a conductive dome array, Furthermore, the external conductive element may be entirely made of conductor material(s), partially made of conductor material(s), or partially made of semiconductor material(s).

In particular, the waterproof housing 60 is provided with at least one slot 62, and at least one hook 112 is provided at the upper surface of the base 110, wherein the at least one hook 112 is configured to be embed into the at least one slot 62 for combination.

In addition, at least two sealing structures (not shown in the drawings) independent from each other can be provided extendedly from the bottom of the waterproof housing 60, wherein each of the sealing structures forms a closed area for accommodating corresponding external conductive elements. Furthermore, the above-mentioned closed areas can be further combined with the upper patch layer 80 or the base 110 to provide waterproof effect in the areas, especially when the above-mentioned closed areas correspond to and closely fit the protruding structures 64 on the base 110.

More particularly, each of the electrodes 100 has an upper surface and a lower surface, wherein a part of the lower surface of each of the electrodes 100 forms a conductive structure, which can be used to contact the skin for sensing the electrocardiogram signals, and a plurality of conductive layers may be formed on the upper surface of each of the electrodes 100; particularly, the conductive layers are not in contact with each other.

The end portion of each of the electrodes 100 is an integrally formed conductive electrode, which can be attached to the skin for sensing the electrocardiogram signals. Furthermore, the end portion of each of the electrodes 100 may also include a conductive electrode and a conductive adhesive combined with each other, wherein the conductive adhesive is sticky and can be used to be attached to the skin as a medium for electrical transmission between the skin and the electrodes 100.

Furthermore, the physiological signal monitoring apparatus according to the second embodiment of the present invention may further include a hollow waterproof ring (not shown in the drawings), which can be used to be combined with the end portion of each of the electrodes 100, and accommodate and contact the conductive adhesive, thereby can prevent the conductive adhesive from directly contacting the upper patch layer 80 or the lower patch layer 90 and affecting the electrical performance of the conductive adhesive, especially when the upper patch layer 80 or the lower patch layer 90 is damp.

Furthermore, the circle diameter of the perforations 81 of the upper patch layer 80 is not greater than 10 mm, and at least a part of the outer edge of the upper patch layer 80 is not smooth.

As described above, the second embodiment of the present invention is characterized in that the waterproof housing being a closed structure itself is combined with the base and then fixed. In particular, the waterproof housing and the upper patch layer are in contact with each other without completely closely fitting, and a plurality of penetration gaps are present therebetween. In addition, the base and the waterproof housing can be combined with each other through hooks and protruding structures of the base, herein, the protruding structures can be a closed foam, rubber or adhesive layer. For example, the hooks made of plastic are perpendicular to the upper patch layer, such that a gap is formed between the surface of the upper patch layer and the bottom of the waterproof housing, which facilitates air permeation and drainage of moisture.

Furthermore, the portion of the upper patch layer extending horizontally beyond the lower patch layer can form an outer edge of the patch layer, which can be completely attached to the skin, and since the upper and lower patch layers of the patch are attached to the skin simultaneously, the effect of prolonging the attachment time can be achieved.

The above descriptions are only provided to explain the preferred embodiments of the present invention, and are not intended to limit the present invention in any form. Therefore, any modifications and changes made without departing from the spirit of the present invention should be included within the scope as claimed in the present invention.

What is claimed is:

1. A physiological signal monitoring apparatus, comprising:
a waterproof housing provided with a bottom opening;
a base located at a bottom of the waterproof housing, the base and the bottom opening of the waterproof housing jointly define a closed waterproof accommodation space;
an electrocardiogram signal processing unit disposed in the waterproof accommodation space;
an upper patch layer having an upper surface and a lower surface, wherein the lower surface of the upper patch layer is sticky, the waterproof housing is located at the upper surface of the upper patch layer, the base is located at the lower surface of the upper patch layer, and a middle area of the upper patch layer is sandwiched by the waterproof housing and the base;
a lower patch layer having an upper surface and a lower surface, wherein the lower surface of the lower patch layer is sticky for being attached to a skin, the lower patch layer is located under the upper patch layer and the base, and the lower patch layer is completely covered by the upper patch layer; and
at least two electrodes located between the upper patch layer and the lower patch layer without contacting with each other, wherein each of the electrodes is electrically coupled, in the waterproof accommodating space, to the electrocardiogram signal processing unit, and penetrates through a joint between the waterproof housing and the base and extends outward in an extension direction, so as to sense an electrocardiogram signal generated by activity of a heart, and transmit the electrocardiogram signal to the electrocardiogram signal processing unit;
wherein all the joint between the waterproof housing and the base overlap the upper patch layer in a vertical direction, and a plurality of parts of the joint overlap the at least two electrodes and the lower patch layer in the vertical direction.

2. The physiological signal monitoring apparatus according to claim 1, wherein the lower patch layer is disposed at a bottom of the base and a bottom of the upper patch layer, so as to be attached and fixed to the skin; and wherein a portion of the lower patch layer disposed at the bottom of the base and a remaining portion of the lower patch layer disposed at the bottom of the upper patch layer are partially located at different levels, or the portion of the lower patch layer disposed at the bottom of the base and the remaining portion of the lower patch layer disposed at the bottom of the upper patch layer are not integrally formed.

3. The physiological signal monitoring apparatus according to claim 1, wherein the upper patch layer is waterproof; a distance of the upper patch layer extending horizontally outward from the waterproof housing is greater than a distance of the lower patch layer extending horizontally outward from the waterproof housing; and a contact area between the upper patch layer and the skin when the upper patch layer is attached to the skin is greater than an area where the at least two electrodes are distributed.

4. The physiological signal monitoring apparatus according to claim 1, wherein each of the at least two electrodes has an end portion extending outward, the lower patch layer is provided with at least two holes respectively corresponding to the end portions, the end portions of the at least two electrodes are in contact with the skin through the at least two holes, respectively, so as to sense the electrocardiogram signal, wherein the holes of the lower patch layer are closely engaged with corresponding end portions respectively to provide waterproof effect, wherein the holes and the corresponding end portions are closely engaged with each other respectively through a double-sided adhesive layer provided therebetween.

5. The physiological signal monitoring apparatus according to claim 1, wherein the upper patch layer is waterproof, and a portion of an outer edge of the upper patch layer extending outward beyond the lower patch layer forms a continuous and uninterrupted area, so as to be completely attached to the skin, thereby forming an area closed with respect to exterior to protect the bottom of the waterproof housing and the lower patch layer; and one of the at least two electrodes only extends outward from an interior of the waterproof housing to an area where the upper patch layer and the lower patch layer overlap with each other, but does not extend to an area where only the upper patch layer is distributed.

6. A physiological signal monitoring apparatus, comprising:
a closed waterproof housing;
an electrocardiogram signal processing unit disposed in the waterproof housing;
an upper patch layer having an upper surface and a lower surface, wherein the lower surface of the upper patch layer is sticky, and the waterproof housing is located at the upper surface of the upper patch layer,
a lower patch layer having an upper surface and a lower surface, wherein the lower surface of the lower patch layer is sticky for being attached to a skin, and the lower patch layer is disposed at the lower surface of the upper patch layer; and
at least two electrodes located between the upper patch layer and the lower patch layer; wherein each of the electrodes is electrically coupled to the electrocardiogram signal processing unit,
wherein a bottom of the waterproof housing and the upper surface of the upper patch layer are combined with each other with at least one gap therebetween, so as to facilitate evaporation of water vapor.

7. The physiological signal monitoring apparatus according to claim 6, wherein the upper patch layer extends horizontally beyond the lower patch layer to form an outer edge of the upper patch layer for being attached to the skin; and the upper patch layer and the lower patch layer have air permeability and moisture permeability, and the air permeability and the moisture permeability of the outer edge of the upper patch layer are greater than those of a portion where the upper patch layer and the lower patch layer are combined with each other.

8. The physiological signal monitoring apparatus according to claim 6, wherein the upper patch layer is provided with a plurality of perforation structures, which are macroscopic and provide the air permeability and the moisture permeability; or the lower patch layer is provided with a plurality of perforation structures, which are macroscopic and provide the air permeability and the moisture permeability.

9. The physiological signal monitoring apparatus according to claim 6, further comprising a base having an upper surface and a lower surface; and the base is disposed between the upper patch layer and the lower patch layer, or on the upper patch layer, or under the lower patch layer, so as to fix and carry the waterproof housing, wherein the base and the waterproof housing are combined in a partially contacting manner.

10. The physiological signal monitoring apparatus according to claim 6, wherein the at least one gap is a penetration gap penetrating through a portion between the bottom of the waterproof housing and the upper surface of the upper patch layer, so as to facilitate evaporation of water vapor.
